(19) United States
(12) Patent Application Publication
(10) Pub. No.: US 2016/0282352 A1
(43) Pub. Date: Sep. 29, 2016

(54) CANCER URINE TEST

(71) Applicant: iSense Medical Corp. (dba Metabolomx), West Palm Beach, FL (US)

(72) Inventors: Raymond Anthony MARTINO, Los Gatos, CA (US); Sung Hyun LIM, Mountain View, CA (US); Paul A. RHODES, Woodside, CA (US)

(21) Appl. No.: 15/034,153

(22) PCT Filed: Nov. 6, 2014
(86) PCT No.: PCT/US2014/064409

§ 371 Date: May 3, 2016

Related U.S. Application Data
(60) Provisional application No. 61/900,875, filed on Nov. 6, 2013.

(51) Int. Cl. G01N 33/574

(52) U.S. Cl. CPC G01N 33/57488; G01N 33/57423; G01N 33/574

(57) ABSTRACT

A urine test for diagnosing lung cancer has been developed that uses an artificial nose such as a colorimetric sensor arrays to identify metabolic profiles in urine headspace gas. Cancer cells excrete unique compounds that are a byproduct of their metabolism. The compounds are excreted through a patient's endocrine system by filtration through the kidneys and other organs and are ultimately excreted through the urine. Some of these cancer cell by-products excrete in the urine are small volatile organic compounds (VOCs). Once the urine has exited the body, these VOCs may be outgassed to the environment. Experimental research has determined that a colorimetric sensor array as disclosed is capable of reliably identifying patients with lung cancer based after exposure to the VOCs of the urine headspace gas.

### FIELD OF THE INVENTION

The present disclosure relates to the diagnosis and screening of cancer using urine.

### BACKGROUND OF THE INVENTION

Lung cancer is the leading cause of cancer related death in the United States. Lung cancer's high mortality rate is partly due to the fact that lung cancer is initially asymptomatic, and therefore not usually detected into later stages of the disease, when intervention and treatment are far less effective. Further, no effective screening test exists to alleviate the death toll from late detection. (Jemal, A., et al., Cancer Statistics, 2010. CA Cancer J. Clin., 2010, 60(5); Sone, S., et al., Long-Term Follow-up Study of a Population-Based 1996-1998 Mass Screening Programme for Lung Cancer Using Mobile Low-Dose Spiral Computed Tomography, Lung Cancer, 2007, 58(3)).

For instance, computed tomography (CT) is currently the gold standard for non-invasive diagnosis of lung cancer, and preferred screening method. However, CT is expensive, and emits significant levels of radiation into the body. For example, the U.S. Preventive Task force recommended in 2013 that roughly 10 million American smokers aged 55 and older should be screened with Low Dose CT annually. The Task Force estimated it would save roughly 20,000 lives a year. However, at roughly $300 a scan, the screen would cost 3 billion dollars to implement annually. Moreover, CT has a false positive rate as high as 19 percent. (Bach, P. B., et al., Benefits and Harms of CT Screening for Lung Cancer A Systematic Review. J. Am. Med. Assoc., 2012, 307(22). For example, the National Lung Screening Trial (NLST), involving 53,454 current and former smokers, found 28% of the high-risk group had a positive CT result, but only 3% of those CT positive patients had lung cancer (Aberle, D. R., et al., Reduced Lung-Cancer Mortality with Low Dose Computed Tomographic Screening. N. Engl. J. Med., 2011. 365(5)). Therefore, CT is quite problematic as a screening tool, as one in five screened subjects may be flagged as potentially having lung cancer. Other available non-invasive screening methods, such as chest X-rays, are even less reliable and are therefore do not provide good alternatives.

Furthermore, confirmation of lung cancer diagnosis usually requires identification of the cancerous cells from the lung tissue, which must either be collected from a biopsy. Given CT's high false positive rate, large numbers of patients are subjected to invasive biopsy tests unnecessarily because no reliable non-invasive method of diagnosing lung cancer exists.

### OBJECTS

It is an object of the disclosure to develop a screening tool for detecting early stage cancers, including lung cancer.

It is an object of the disclosure to improve the accuracy and reduce false positives in diagnosing lung cancer.

It is an object of the disclosure to reduce the cost involved with testing for lung cancer.

It is an object of the disclosure to reduce health care spending through early identification and treatment of cancer.

It is an object of the disclosure to develop a more accurate non-invasive diagnostic test for lung cancer.

It is an object of the disclosure to develop a non-invasive diagnostic test for identifying the subtype of lung cancer.

### SUMMARY

A urine test for diagnosing lung cancer has been developed that uses an artificial nose such as a colorimetric sensor arrays to identify metabolic profiles in urine headspace gas. Cancer cells excrete unique compounds that are a byproduct of their metabolism. The compounds are excreted through a patient's endocrine system by filtration through the kidneys and other organs and are ultimately excreted through the urine. Some of these cancer cell by-products excrete in the urine are small volatile organic compounds (VOCs). Once the urine has exited the body, these VOCs may be outgassed to the environment.

These cancer cell VOCs provide a potential biomarker for the identification of patients with cancer. However, these cancer cell metabolism byproducts are excreted along with byproducts of the remainder of the body's somatic cells, and any other VOCs that result from the breakdown of the food and drink ingested by the body. Accordingly, the profile of VOCs outgassed from urine is diverse and crowded, and contains numerous VOCs excreted from somatic cells or that are a byproduct of digestion along with VOCs excreted from lung cancer cells. Therefore, developing an instrument that is able to reliably identify the unique VOC profile of cancer or lung cancer in the urine of a patient among the plethora of other VOCs present is quite challenging.

VOC selective detectors or “artificial noses” have developed to detect and characterize gaseous samples. A multitude of technologies have implemented artificial nose functions including, but not limited to: colorimetric sensor arrays, polymer arrays, mass sensitive piezoelectric substrates, surface acoustic wave (SAW) transducers, quartz crystal microbalances, functionalized carbon nanotubes and gold nano-particles.

Initial work in the field of artificial noses was conducted by Wilkens and Hatman in 1964, though the bulk of research done in this area has been carried out since the early 1980's. See, e.g., W. F. Wilkens, A D. Hatman. Ann. NY Acad. Sci., 116, 608 (1964); K. Pursaud, G. H. Dodd. Nature, 299, 352-355 (1982); and J. W. Gardner, P. N., Bartlett. Sensors and Actuators B, 18-19, 211-220 (1994). Vapor-selective detectors or “artificial noses” are typically based upon the production of an interpretable signal or display upon exposure to a vapor emitting substance or odorant (hereinafter sometimes referred to as an “analyte”). More specifically, typical artificial noses are based upon selective chemical binding or other molecular interactions in the interface between a detecting compound of the artificial nose and an analyte or odorant, and then transforming that chemical binding into a signal or display, i.e., signal transduction.

Polymer arrays having a single dye have been used for artificial noses. That is, a series of chemically-diverse polymers or polymer blends are chosen so that their composite response distinguishes a given odorant or analyte from others. Examples of polymer array vapor detectors, including conductive polymer and conductive polymer/carbon black composites, are discussed in: M. S. Freund, N. S. Lewis, Proc. Natl. Acad. Sci. USA 92, 2652-2656 (1995); B. J. Doleman, R. D. Sanner, E. J. Severin, R. H. Grubbs, N. S. Lewis, Anal. Chem. 70, 2560-2564 (1998); T. A Dickinson, J. White, J. S. Kauer, D. R. Walt, Nature 382, 697-700 (1996)(polymer array with optical detection); A E. Hoyt, A J. Ricco, H. C. Yang, R. M. Crooks, J. Am. Chem. Soc. 117,8672 (1995); and J. W. Grate, M. H. Abraham, Sensors and Actuators B 3, 85-111 (1991).

Other interface materials include functionalized self-assembled monolayers (SAM), metal oxides, and dendrimers. Signal transduction is commonly achieved with mass sensitive piezoelectric substrates, surface acoustic wave (SAW) transducers, or conductive materials. Optical transducers (based on absorbance or luminescence) have also been examined. Examples of metal oxide, SAM, and dendrimer-based detectors are discussed in J. W. Gardner, H. V. Shurmer, P. Corcoran, Sensors and Actuators B 4, 117-121(1991); J. W. Gardner, H. V. Shurmer, T. T. Tan, Sensors and Actuators B 6, 71-75 (1992); and R. M. Crooks, A. J. Ricco, Acc. Chem. Res. 31, 219-227 (1998). These devices also use a single dye.

Techniques have also been developed using a metalloporphyrin for optical detection of a specific, single gas such as oxygen or ammonia, and for vapor detection by chemically interactive layers on quartz crystal microbalances. See A. E. Baron, J. D. S. Danielson, M. Gonterman, J. R. Wan, J. B. Callis, Rev. Sci. Instrum. 64, 3394-3402 (1993); J. Kavandi, et al., Rev. Sci. Instrum. 61, 3340-3347 (1990); W. Lee, et al., J. Mater. Chem. 3, 1031-1035 (1993);A. A. Vaughan, M. G. Baron, R. Narayanaswamy, Anal Comm. 33, 393-396 (1996); J. A J. Brunink, et al., Anal. Chim. Acta 325, 53-64 (1996); C. DiNatale, et al., Sensors and Actuators B 44, 521-526 (1997); and C. DiNatale, et al., Mat. Sci. Eng. C 5, 209-215 (1998).

Other techniques include functionalized carbon nanotubes sometimes integrated into a transistor, see DNA-Decorated Carbon Nanotubes for Chemical Sensing Cristian Staii and Alan T. Johnson, Jr, Nano Letters 2005 and functionalized gold nanoparticles see Broza, Y. Y., & Haick, H. (2013). Nanomaterial-based sensors for detection of disease by volatile organic compounds. Nanomedicine, 8(5), 785-806 ; Barash, O., Peled, N., Hirsch, F. R., & Haick, H. (2009). Sniffing the Unique “Odor Print” of Non-Small-Cell Lung Cancer with Gold Nanoparticles. Small, 5(22), 2618-2624.

Artificial noses based on colorimetric sensor arrays exist that are capable of detecting VOCs at low concentrations and a high degree of accuracy. Colorimetric sensor arrays that are capable of detecting VOCs typically contain chemically responsive dyes that change color when VOCs contact the dye molecules, and cause a chemical reaction. Sensor arrays typically contain a variety of types of reactive molecules that respond to different VOCs. Examples of sensor arrays are described in, for example, U.S. Pat. No. 6,368,558, issued on Apr. 9, 2002, titled Colorimetric Artificial Nose Having an Array of Dyes and Method for Artificial Olfaction, and Lim et al, An optoelectronic nose for the detection of toxic gases. Nature Chemistry, 10.1038, 564-567, 2009, both of which are incorporated by reference herein in their entirety. Accordingly, when exposed to a certain mixture of VOCs, an array of chemo-responsive dyes will change color in a distinct pattern that will be distinguishable from the color change using a different VOC mixture. Thus, with a large enough sensor array that includes a sufficient number of types of chemo-responsive dyes, a fingerprint of the VOCs contained in a particular patient's urine headspace gas can be detected.

Despite the plethora of VOCs in the urine headspace gas, experimental research has determined that a colorimetric sensor array as disclosed is capable of reliably identifying patients with lung cancer based after exposure to the urine headspace gas. Thus, the present disclosure provides a cost effective, accurate, and non-invasive test for screening and diagnosing patients with lung cancer. Additionally, experimental research has determined that the colorimetric sensor array as disclosed is capable of identifying the subtype of cancer in patients. Accordingly, the disclosed devices and methods have the potential to tens of thousands of lives in the United States annually and also provide earlier and more effective treatment for patients with lung cancer. This will lead to better quality of life, lower healthcare costs, for the United States, based on the diagnosis and screening of a disease that has remained elusive.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 is an overview of a system implementing an embodiment of a urine test for cancer diagnosis according to the present disclosure;

FIG. 2A is illustrates an embodiment of a container in accordance with the present disclosure;

FIG. 2B is illustrates an inside of an array portion of a container with a spacer, cover sheet, and sensor array.

FIG. 2C is a perspective view of a container with an array portion removed to reveal an absorbent layer and septa or sealing ring in accordance with the present disclosure;

FIG. 2D is a perspective view of a stopper in accordance with the present disclosure;

FIG. 3A is a perspective view of a container with a stopper inserted in an opening of the container in accordance with the present disclosure

FIG. 3B is a perspective view of a container with the stopper removed to reveal a sample opening in accordance with the present disclosure.

FIG. 4 is an exploded view of an embodiment of a container in accordance with the present disclosure.

FIG. 5 is an exploded view of an embodiment of a container including a pre-oxidation channel in accordance with the present disclosure.

FIG. 6A is a perspective view of an embodiment of a container with a spiral oxidation chamber in accordance with the present disclosure.

FIG. 6B is an exploded view of an embodiment of a container with a spiral oxidation chamber in accordance with the present disclosure.

FIG. 7 is an embodiment of a colorimetric sensor array in accordance with the present disclosure.

FIG. 8 illustrates an embodiment of a process for outputting an indication of a likelihood a patient has cancer in accordance with the present disclosure.

FIG. 9A is an illustration of a colorimetric sensor array prior to exposure to the sample in accordance with the present disclosure.

FIG. 9B is an illustration of a colorimetric sensor array following to exposure to the sample in accordance with the present disclosure.

FIG. 9C is an illustration of a colorimetric representation of the color change between FIG. 9A and FIG. 9B.

FIGS. 10A-10C are plots of a principle component analysis (PCA) performed on the experimental results of an example implementing the devices and methods of the present disclosure.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

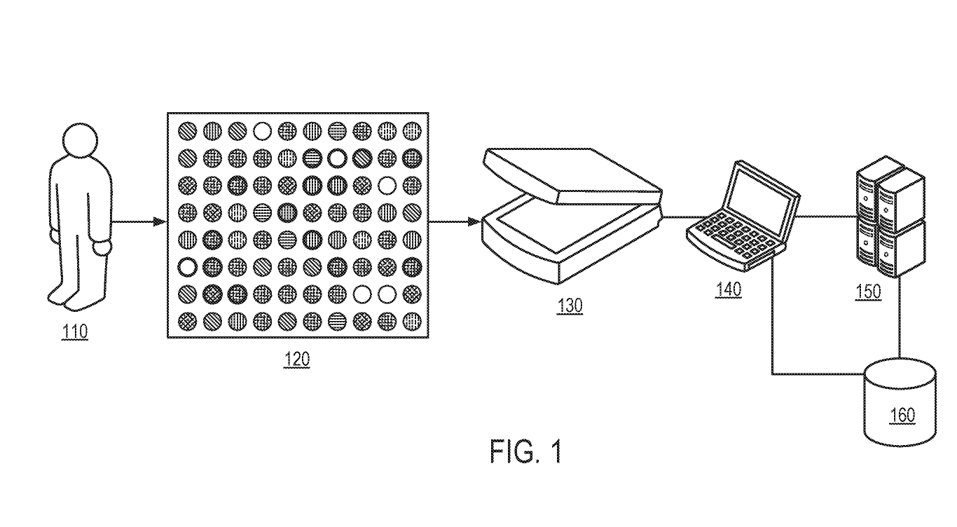

FIG. 1 is an overview of systems and methods that may be implemented for performing a urine test to diagnose of cancer. First, a patient 110 may provide a urine sample to a caregiver for testing and analysis. The caregiver may then bring gas from the urine sample into gaseous contact with a colorimetric sensor array 120 or other artificial nose technology. The gas may be applied to the colorimetric sensor array using any suitable configuration, vial, or container in order to allow the sample headspace gas to contain the colorimetric sensor array 120. In some examples, the sensor array 120 may be scanned or imaged by a detector 130 prior to exposure of the gas to the array in order to establish a baseline from which to determine a color change. If alternative artificial nose technology is employed, a similar baseline reading of the artificial nose may be taken prior to introduction of the sample. Following the caregiver's exposure of the headspace gas to the array, a detector 130 may take an image or record a data reading at a single time following application, or at several times following application. The data from the detector 130 or other output from an artificial nose may then be processed by a processing device 140 or sent to a server 150 for processing to determine whether a patient 110 has lung cancer, and in some embodiments, the specific strain of lung cancer.

In some embodiments, the processing device 140 will determine the color change based on the differences in images of a sensor array 120 or other artificial nose technology taken before and after exposure to a sample. Using this data, in some embodiments, the system may determine a unique fingerprint for the patient sample. Then the sample can be compared to a database 160 of prior data of patients with non-diseases, including lung cancer. In some embodiments, simple statistical analysis such as PCA and HCA may be performed to determine whether the sample color change indicates the patient is likely to have lung cancer. The system may then output the results from the server 150 or processing device 140 for display to the caregiver or patient 110. The results of the processing may indicate a probability or flag indicating the patient 110 has one of several diseases.

### Sample Container

The urine headspace gas may be exposed to the sensor array 120 or other artificial nose technology in a variety of methods and devices. In some embodiments, the sample urine is deposited in a container that has an airtight seal and minimizes the exposure of the sensor array 120 to contaminants other than the urine headspace gas.

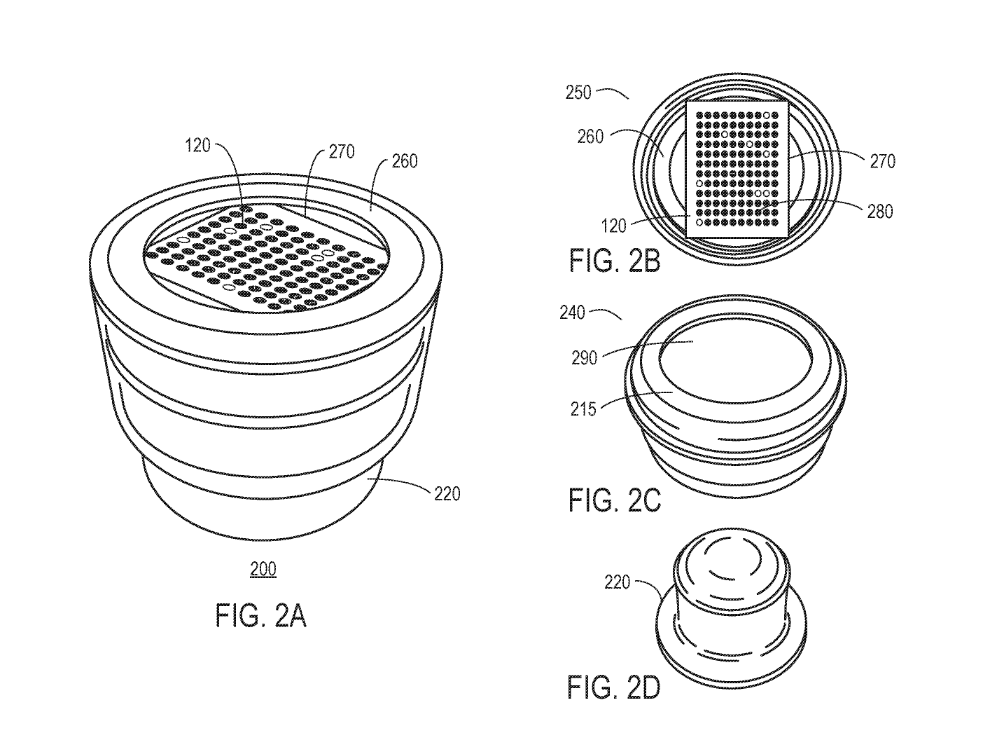

FIGS. 2A-2C illustrate an example of a sample container 200 that contains a colorimetric sensor array 120 for exposure to urine headspace gas. In other embodiments, a container 200 may be used to expose a sensor array 200 to other biological fluids for detection of VOCs outgassed by the fluids. In some embodiments, other types of artificial nose technology as disclosed herein may be used in place of a colorimetric sensor array 120 in order to detect the VOCs outgassed by the fluids. A container 200 in connection with the present disclosure may be any suitable container that limits airflow or provides a cavity to allow the headspace gas from the sample to be directed towards and exposed to a colorimetric sensor array 120. This may include vials, tubes, channels, ducts, or separate chambers that are later mixed or brought into vicinity. The sample container 200 may be prefabricated with the sensor array 120 inside. In other examples, the container 200 may separate or have an opening to allow an array 120 to be inserted and removed. The container 200 may contain a transparent portion or other window 270 to allow a detector 130 to scan images of a colorimetric sensor array 120. In some embodiments, the window may filter out certain wavelengths to improve the quality of signal and image. For example, the window 270 may contain a polarized filter to filter out unwanted noise from the signal.

The sample container 200 may contain a sample portion 240 and an array portion 250. The sample portion 240 and array portion 250 may be removably connectable through any suitable methods or devices known in the art, including screw systems, snap systems, or welding. The sample container 200 may be fabricated from any suitable inert material, including a variety of plastics. The sample container 200 may be fabricated from a material that exhibits minimal outgassing of VOCs or other contaminants that may interfere or react with the sensor array 120.

FIG. 2B illustrates an embodiment of an array portion 250 of a container 200. The array portion 250 may include a window 270 for imaging of the sensor array 120. One the side of the sensor array 120 adjacent to the window 270, a spacer 260 may be included to allow a space between the sensor array 120 and window 270 to permit the urine gas VOCs to contact and react with the reactive side of the sensor array 120. In some examples, a cover 280 or other obstructive structure or catch tray may be placed on the non-window 270 side of a sensor array 120 to prevent urine from dripping onto the array 120 while the sample and array 120 are reacting.

FIG. 2C illustrates an embodiment of a sample portion 240 of a container 200. The sample portion 240 may contain an absorbent layer 290 for application and absorption of the urine sample. A sealing ring 215 or other washer or sealing device may be applied on top of or around the edges of absorbent layer 290. Once the sample portion 240 is fixed to the array portion 250, the sealing ring 215 is pressed tightly against the absorbent layer 290, to prevent airflow around the edges of the absorbent layer 290. This is advantageous because otherwise contaminating air may reach the sensor array 120 during application of the sample. As shown in FIG. 2D, the sample container 200 may contain a stopper 220 that is removably inserted in an opening in a sample portion 240 for creating an airtight seal inside of a sensor tube 200.

In order to apply a urine sample, a caregiver may remove a stopper 200 from the opening in the sample portion 240, and deposit a small amount of the liquid urine on the absorbent layer 290. The absorbent layer 290 is ideally a material that will absorb a certain amount of urine and allow the urine to soak through to the opposite side of the absorbent layer 290 and outgas VOCS into the headspace of the container 200. The absorbent layer 290 may be a disk or flat paper or may be different shaped including a ball, ellipse or other suitable shapes. In some embodiments, the absorbent layer 290 may be fibrous or porous to allow maximal surface area for the urine to outgas VOCs. In other embodiments, the absorbent layer may include capillaries that allow the urine to travel to the other side and allow VOCs to offgas. After the sample excretes VOCs, the gaseous VOCs may then contact the sensor array 120.

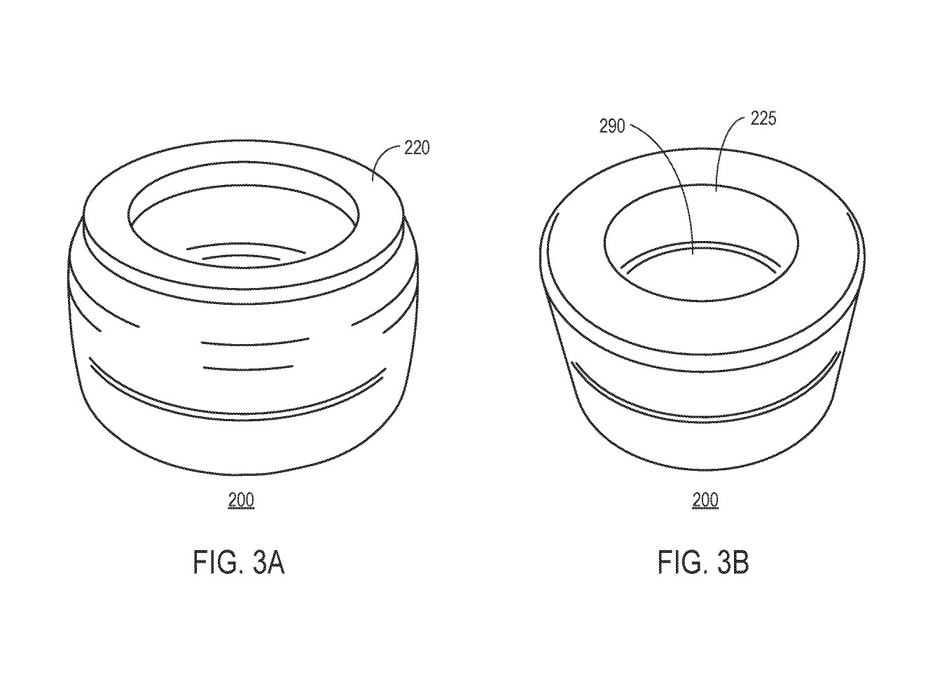

FIG. 3A illustrates an embodiment of a sample container 200, with the sample portion and stopper 220 facing up. This view illustrates the stopper 220 inserted securely inside the sample container 200 in order to form a seal around the edges of the stopper 220 and trap the headspace gas from the urine inside the container 200. The stopper 200 may be removed from the bottom of the sensor tube 200 in order to place a sample of urine inside the sensor tube 200.

FIG. 3B illustrates an embodiment of a sample container 200, with the sample potion 240 facing up. This view illustrates the stopper 220 removed to allow access to the internal cavity through the sample opening 225. A caregiver may remove stopper 220 in order to deposit a urine sample or other biological sample on the absorbent material or layer 290.

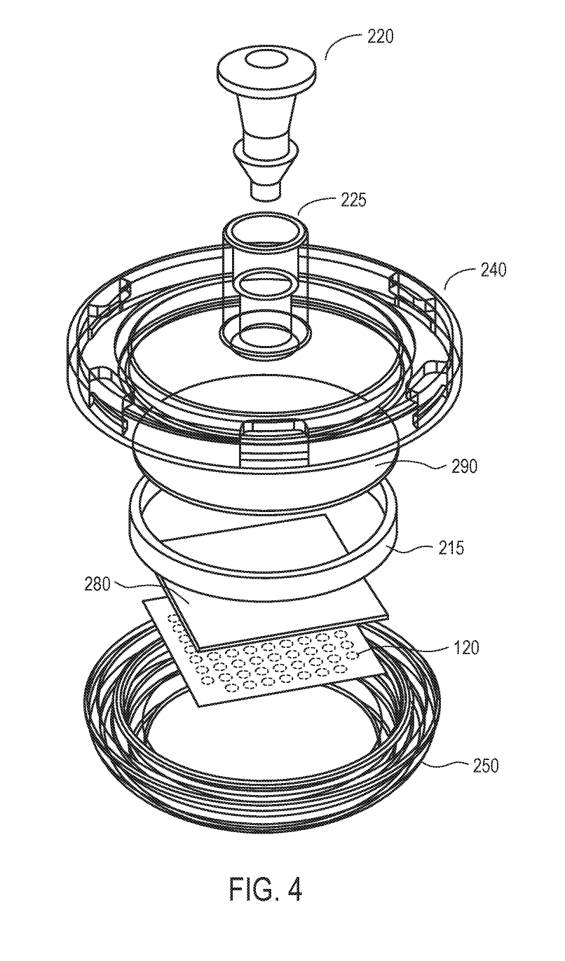

FIG. 4 is an exploded view of an embodiment of a container 200. The container 200 contains a sample portion 240 and an array portion 250. In some embodiments, the sample portion 240 and array portion 250 snap, screw, or are welded together to form an airtight seal to prevent gas or other molecules for entering or exiting the compartment once sealed. FIG. 4 illustrates an embodiment in which the sample portion 240 snaps to affix to the array portion 250. FIG. 4 illustrates an embodiment of an absorbent layer 290 in a disc shape. The absorbent layer 290 may be any suitable absorbent material, for instance blotting paper that would absorb urine while simultaneously allow outgassing of urine VOCs. In some embodiments, the absorbent layer 290 must be thin enough to allow a small sample of urine to absorb through to the opposite side of the absorbent layer 290. In other embodiments, the absorbent layer 290 may be thicker to prevent less air diffusion through absorbent layer 290 or to allow a larger urine sample to be applied to absorbent layer 290.

The sample portion 240 may contain a sample opening 225 for application of a urine sample to the absorbent layer 290. To apply a sample, a caregiver may remove a stopper 220 from a sample opening 225 and a urine sample may be deposited on the absorbent layer 290. The stopper 220 may then be immediately replaced to form an airtight seal and prevent contaminants from the environment from entering the container 200. Then, the urine may soak through to the other side of the absorbent layer 290 and/or begin to release VOCs into the headspace of the container 200. The sealing ring 215 would prevent the urine or outside air from entering the airspace of the array portion 250 of the container 200. The VOCs may then begin to react with the sensor array 120 and change the array 120 color.

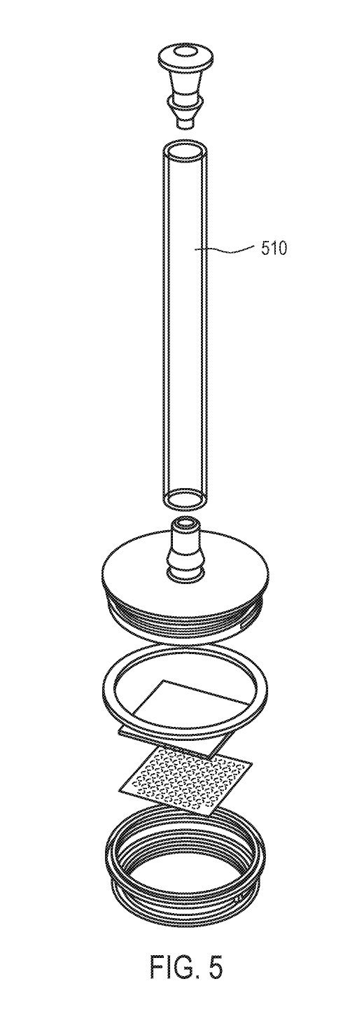

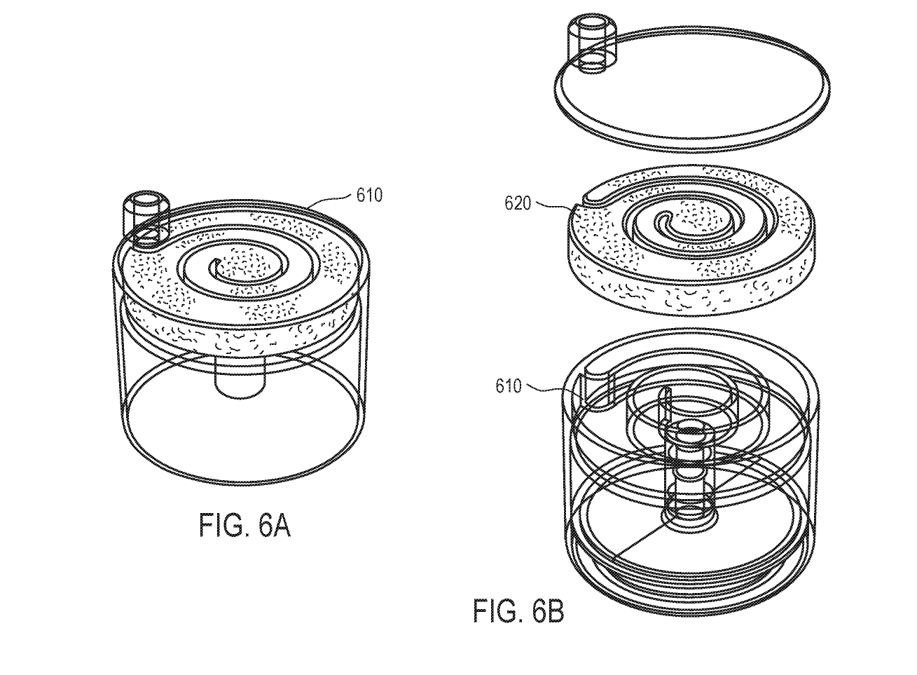

FIG. 5 illustrates another embodiment of an exploded view of a sample container 200 that incorporates pre-oxidation channel 510 for samples that require oxidation. In some embodiments, oxidation of samples may improve accuracy or specificity of identification or diagnosis of samples. FIGS. 6A and 6B illustrate another embodiment of a sensor tube 200 that has a spiral oxidation chamber 610 and oxidation insert 620. After a caregiver applies a urine sample to a container 200, the urine will begin to outgas VOCs that will contact the colorimetric sensor array 120 which will begin to react and change color depending on the VOCs contained in the urine sample.

### Artificial Nose

VOC selective detectors or “artificial noses” have developed to detect and characterize gaseous samples. A multitude of technologies have implemented artificial nose functions including, but not limited to: colorimetric sensor arrays, polymer arrays, mass sensitive piezoelectric substrates, surface acoustic wave (SAW) transducers, quartz crystal microbalances, functionalized carbon nanotubes and gold nano particles.

Initial work in the field of artificial noses was conducted by Wilkens and Hatman in 1964, though the bulk of research done in this area has been carried out since the early 1980's. See, e.g., W. F. Wilkens, A D. Hatman. Ann. NY Acad. Sci., 116, 608 (1964); K. Pursaud, G. H. Dodd. Nature, 299, 352-355 (1982); and J. W. Gardner, P. N., Bartlett. Sensors and Actuators B, 18-19, 211-220 (1994). Vapor-selective detectors or “artificial noses” are typically based upon the production of an interpretable signal or display upon exposure to a vapor emitting substance or odorant (hereinafter sometimes referred to as an “analyte”). More specifically, typical artificial noses are based upon selective chemical binding or other molecular interactions in the interface between a detecting compound of the artificial nose and an analyte or odorant, and then transforming that chemical binding into a signal or display, i.e., signal transduction.

Polymer arrays having a single dye have been used for artificial noses. That is, a series of chemically-diverse polymers or polymer blends are chosen so that their composite response distinguishes a given odorant or analyte from others. Examples of polymer array vapor detectors, including conductive polymer and conductive polymer/carbon black composites, are discussed in: M. S. Freund, N. S. Lewis, Proc. Natl. Acad. Sci. USA 92, 2652-2656 (1995); B. J. Doleman, R. D. Sanner, E. J. Severin, R. H. Grubbs, N. S. Lewis, Anal. Chem. 70, 2560-2564 (1998); T. A Dickinson, J. White, J. S. Kauer, D. R. Walt, Nature 382, 697-700 (1996) (polymer array with optical detection); A E. Hoyt, A J. Ricco, H. C. Yang, R. M. Crooks, J. Am. Chem. Soc. 117,8672 (1995); and J. W. Grate, M. H. Abraham, Sensors and Actuators B 3, 85-111 (1991).

Other interface materials include functionalized self-assembled monolayers (SAM), metal oxides, and dendrimers. Signal transduction is commonly achieved with mass sensitive piezoelectric substrates, surface acoustic wave (SAW) transducers, or conductive materials. Optical transducers (based on absorbance or luminescence) have also been examined. Examples of metal oxide, SAM, and dendrimer-based detectors are discussed in J. W. Gardner, H. V. Shurmer, P. Corcoran, Sensors and Actuators B 4, 117-121(1991); J. W. Gardner, H. V. Shurmer, T. T. Tan, Sensors and Actuators B 6, 71-75 (1992); and R. M. Crooks, A. J. Ricco, Acc. Chem. Res. 31, 219-227 (1998). These devices also use a single dye.

Techniques have also been developed using a metalloporphyrin for optical detection of a specific, single gas such as oxygen or ammonia, and for vapor detection by chemically interactive layers on quartz crystal microbalances. See A. E. Baron, J. D. S. Danielson, M. Gonterman, J. R. Wan, J. B. Callis, Rev. Sci. Instrum. 64, 3394-3402 (1993); J. Kavandi, et al., Rev. Sci. Instrum. 61, 3340-3347 (1990); W. Lee, et al., J. Mater. Chem. 3, 1031-1035 (1993);A. A. Vaughan, M. G. Baron, R. Narayanaswamy, Anal Comm. 33, 393-396 (1996); J. A J. Brunink, et al., Anal. Chim. Acta 325, 53-64 (1996); C. DiNatale, et al., Sensors and Actuators B 44, 521-526 (1997); and C. DiNatale, et al., Mat. Sci. Eng. C 5, 209-215 (1998).

Other techniques include functionalized carbon nanotubes sometimes integrated into a transistor, see DNA-Decorated Carbon Nanotubes for Chemical Sensing Cristian Staii and Alan T. Johnson, Jr ,Nano Letters 2005 and functionalized gold nanoparticles see Broza, Y. Y., & Haick, H. (2013). Nanomaterial-based sensors for detection of disease by volatile organic compounds. Nanomedicine, 8(5), 785-806 ; Barash, O., Peled, N., Hirsch, F. R., & Haick, H. (2009). Sniffing the Unique “Odor Print” of Non-Small-Cell Lung Cancer with Gold Nanoparticles. Small, 5(22), 2618-2624.
Colorimetric Sensor Arrays

Artificial noses based on colorimetric sensor arrays exist that are capable of detecting VOCs at low concentrations and a high degree of accuracy. Colormetric sensor arrays 120 may detect volatile organic compounds by reacting with the compounds and changing color based on the amount and type compounds exposed to the array 120. The resulting pattern of color changes comprises a high-dimensional fingerprint which enables the identification of complex mixtures, including disease signatures in exhaled breath and in sealed assays. Various colorimetric sensor arrays are described in the following patent publications to Suslick et al. and all of which are incorporated by reference herein in their entirety: U.S. Pat. No. 6,368,558 to Suslick, U.S. Pat. No. 6,495,102, to Suslick, et al., U.S. Pat. No. 7,261, 857, to Suslick et al., and U.S. Patent Publication 2008/0199904.

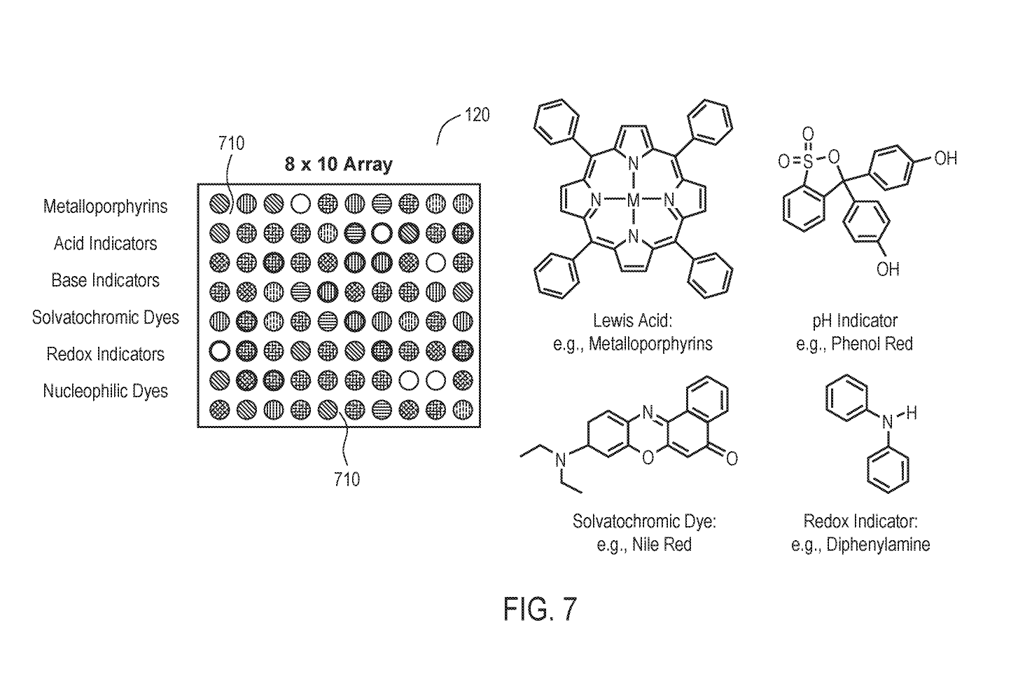

FIG. 7 illustrates an embodiment of a colorimetric sensor array 120. In an embodiment, a colorimetric sensor array 120 may include a substrate 720 upon which a variety of chemically responsive dyes 710 may be deposited. The dyes 710 may change color after exposure to and reacting with volatile organic compounds. Certain dyes are responsive to certain VOCs allowing for a particular mixture of VOCs to be determined by its unique color change exhibited on the sensor array 120.
Chemo-Responsive Dyes

Colorimetric sensor arrays utilizing chemo-responsive (chemically responsive) dyes 710 are capable of detecting individual VOC's and complex VOC mixtures down to low part per billion (ppb) concentrations [10, 11, 25]. For example, the following five classes of chemically-responsive dyes may be utilized: (i) metal-ion-containing dyes that respond to Lewis basicity (i.e., electron pair donation, metal ion ligation), (ii) pH indicators that respond to Brønsted acidity/basicity (i.e., proton acidity and hydrogen bonding), (iii) dyes with large permanent dipoles (e.g., solvatochromic dyes) that respond to local polarity, (iv) metal salts that respond to redox reactions, and (v) nucleophilic indicators that respond to electrophilic analytes. Utilizing of this broad spectrum of highly sensitive chemical interactions allows a colorimetric sensor array 120 to detect and identify very diverse classes of metabolite compounds.

For example, for recognition of analytes with Lewis acid/base capabilities, the use of porphyrins and their metal complexes is desirable Metalloporphyrins are ideal for the detection of metal-ligating vapors because of their open coordination sites for axial ligation, their large spectroscopic shifts upon ligand binding, their intense coloration, and their ability to provide ligand differentiation based on metal-selective coordination, Furthermore, metalloporphyrins are cross-responsive dyes, showing responses to a large variety of different analytes to different degrees and by different color changes.

A Lewis acid/base dye is defined as a dye which has been identified for its ability to interact with analytes by acceptor-donor sharing of a pair of electrons from the analyte. This results in a change in color and/or intensity of color that indicates the presence of the analyte. Lewis acid/base dyes include metal ion-containing or three-coordinate boron-containing dyes.

Exemplary Lewis acids include, but are not limited to, metal ion-containing porphyrins (i.e., metalloporphyrins), salen complexes, chlorins, bispocket porphyrins, and phthalocyanines. Particularly suitable metal ions complexed with dyes for detecting ammonia include Zn(II) and Co(III) metals. In particular embodiments of the present invention, the Lewis acid dye is a metalloporphyrin. For example, diversity within the metalloporphyrins can be obtained by variation of the parent porphyrin, the porphyrin metal center, or the peripheral porphyrin substituents. The parent porphyrin is also referred to as a free base porphyrin, which has two central nitrogen atoms protonated (ie., hydrogen cations bonded to two of the central pyrrole nitrogen atoms). A particularly suitable parent porphyrin is 5,10,15,20-tetraphenylporphyrinate(-2) (TPP dianion), its metalated complexes, its so-called free base form (H₂FPP) and its acid forms (H₃TPP⁺ and H₄TPP⁺²). Suitable metal ion-containing metalloporphyrin dyes for use in the apparatus and method of the present invention include, but are not limited to, 2,3,7,8,12,13,17,18-octafluoro-5,10,15,20-tetrakis-(pentafluorophenyl)porphyrinatocobalt(II) [Co(F₂₈TPP)];

2,3,7,8,12,13,17,18-octabromo-5,10,15,20-tetrapheny porphyrinatozine(II) [Zn(Br₈TPP)];

5,10,15,20-tetraphenylporphyiinatozine(II) [ZnTPP];

5(phenyl)-10,15,20-trikis(2′,6′-bis(dimethyl-t-butylsiloxyl)phenyl) porphyrinatozine(II) [Zn(Si₆PP)]

5,10,15,20-tetrakis(2′,6′-bis(dimethyl-t-butysiloxyl)phenyl)porphyrinatozine(II) [Zn (Si₈PP];

5,10,15,20-Tetraphenyl -porphyrinatocobalt (II) [CoTPP];

5,10,15,20-Tetrakis(2,6-difluorophenyl)porphyrinatozine(II) [Zn—F2PP]; and

5,10,15,20-Tetrakis(2,4,6-trimethylphenyl)porphyrinatozinc(II) [ZnTMP].

The synthesis of such porphyrins is described in U.S. patent application Ser. No. 10/279,788.

A Bronsted acid dye of the present disclosure is a pH indicator dye which changes color in response to changes in the proton (Bronsted) acidity or basicity of the environment. For example, Bronsted acid dyes are, in general, non-metalated dyes that are proton donors which can change color by donating a proton to a Bronsted base (i.e., a proton acceptor). Bronsted acid dyes include, but are not limited to, protonated, but non-metalated, porphyrins, chlorins, bispocket porphyrins, phthalocyanines, and related polypyrrolic dyes. Polypyrrolic dyes, when protonated, are in general pH-sensitive dyes (i.e., pH indicator or acid-base indicator dyes that change color upon exposure to acids or bases) In one embodiment, a Bronsted acid dye is a non-metalated porphyrin such as 5,10,15,20-tetrakis(2′,6′-bis(dimethyl-t-butylsiloxyl)phenyl)porphyrin dication [H₄Si₈PP]⁺²; 5,10,15,20-Tetraphenyl-21H,23H-porphine [H₂TPP]; or 5,10,15,20-Tetraphenylporphine dication [H₄TPP]⁺². In another embodiment of the instant invention, a selected Bronsted dye is an indicator dye including, but not limited to, Bromocresol Purple, Cresol Red, Congo Red, Thymol Blue, Bromocresol Green, Nile Red, Bromothymol Blue, Methyl Red, Nitrazine Yellow, Phenol Red, Bromophenol Red, Disperse Orange 25, and Bromophenol Blue. As will be appreciated by the skilled artisan, the Bronsted acids disclosed herein may also be considered Bronsted bases under particular pH conditions. Likewise, a non-metalated, non-protonated, free base form of a bispocket porphyrin may also be considered a Bronsted base. However, these dye forms are also expressly considered to be within the scope of the dyes disclosed herein.

Solvatochromic dyes that may be utilized change color in response to changes in the general polarity of their environment, primarily through strong dipole-dipole and dispersion interactions. To some extent, all dyes inherently are solvatochromic, with some being more responsive than others. Particular examples of suitable solvatochromic dyes include, but are not limited to Reichardt's dyes, 4-hydroxystyryl-pyridinium dye, 4-methoxycarbonyl-1-ethylpyridinium iodide, and 2,6-diphenyl-4-(2,4,6-triphenyl-1-pyridinio)-phenolate.

The addition of at least one Bronsted acid dye to an array 120 containing at least one metal ion-containing Lewis acid dye can improve the sensitivity of the array 120 for particular analytes and increase the ability to discriminate between analytes. For example, colorimetric sensor arrays 120 have been shown to detect volatile organic compounds and complex mixtures down to ppb levels (Rakow, et al. (2005) Angew. Chem. Int. Ed. 44:4528-4532). Further, the use of one or more metal ion-containing dyes in combination with one or more Bronsted acid dyes can advantageously create a signature indicative of the presence of a particular analyte. Thus, while some embodiments may utilize at least one Lewis acid and/or base dye, one Bronsted acidic and/or basic dye, or one zwitterionic solvatochromic dye, other embodiments of this disclosure may utilize use at least two different classes of dyes on the instant arrays 120. In one embodiment, the colorimetric sensor array 120 contains at least one Lewis acid and/or base dye, one Bronsted acidic and/or basic dye, or one zwitterionic solvatochromic dye. In another embodiment, the colorimetric sensor array 120 contains at least one Lewis acid and/or base dye and one Bronsted acidic and/or basic dye. In a further embodiment, the colorimetric sensor array 120 contains at least one Lewis acid and/or base dye and one zwitterionic solvatochromic dye. In yet a further embodiment, the colorimetric sensor array 120 contains at least one Bronsted acidic and/or basic dye and one zwitterionic solvatochromic dye. Still further embodiments may utilize at least three different classes of dyes on the instant arrays, i.e., at least one Lewis acid and/or base dye, one Bronsted acidic and/or basic dye, and one zwitterionic solvatochromic dye
Dye Substrate

In accordance with the present invention, the plurality of chemo-responsive dyes 710 may be deposited on an array substrate 720 in a predetermined pattern combination. Alternatively stated, the dyes 710 are arranged in a two-dimensional (or linear or other arrangement) spatially-resolved configuration so that upon interaction with one or more analytes, a distinct color and intensity response of each dye creates a signature indicative of the one or more analytes. A plurality of chemo-responsive dyes encompasses 2, 3, 4, 5, 6, 7, 8, 9, 10, 15, 20, 25, 30, 35, 40, or 50 individual dyes. In particular embodiments, a plurality of chemo-responsive dyes is 2 or more, 5 or more, 10 or more, 15 or more, 20 or more, 25 or more, or 30 or more dyes. The chemo-responsive dyes can be deposited in predetermined pattern combinations of rows, columns, spirals, etc., and one or more chemo-responsive dye arrays can be used in a container. Dyes 710 can be covalently or non-covalently affixed in or on a colorimetric sensor array 120 substrate 720 by direct deposition, including, but not limited to, airbrushing, ink-jet printing, screen printing, stamping, micropipette spotting, or nanoliter dispensing.

The substrate 720 for retaining the chemo-responsive dyes 710 may be any suitable material or materials, including but not limited to, chromatography plates, paper, filter papers, porous membranes, or properly machined polymers, glasses, or metals. In some embodiments, the substrate 720 may include a hydrophobic substrate. In some embodiments, a nanoporous sol-gel matrix is used as a substrate 720 for the dyes 710.

A nano-porous pigment may be fabricated by the immobilization of chemically responsive dyes in organically modified siloxanes (ormosils). In some embodiments, the pigment is created by utilizing an electronic spray to generate an aerosol from precursor solutions containing the dye 710 and other materials, which is then heated to form dye encapsulated microspheres. These dye-encapsulated microspheres can then be printed on paper, such as chromatography paper to form a colorimetric sensor array 120. These porous sol-gel ormosils may provide a good matrix for colorants due to high surface area, good stability over a wide range of pH, relative inertness in many environments, and transparency in the UV-visible spectrum.

A nanoporous sol-gel matrix has enormous surface area at a microscopic scale, which results in the part-per-billion (ppb) sensitivity. In some embodiments, a nanoporous sol-gel matrix may be required to detect the trace volatile organic compound (VOC) signatures of lung cancer and other diseases in urine or other biological fluids. The nanoporous pigment is a silicon-based sol-gel with enormous surface area, vastly increasing interaction opportunities between analyte and indicator and thereby achieving great sensitivity across a wide range of volatile molecules, including species crucial for cancer diagnosis. Furthermore, the high chemical resistance of the nanoporous pigment allows a manufacturer to increase the chemical diversity of the dyes 710 deposited on the nanoporous substrate 720 that were by adding chromogenic reagents that were too reactive to incorporate onto substrates 720 comprised of different materials. Nanoporous pigments are more fully described in Lim et al., Chemically Responsive Nanoporous Pigments: Colorimetric Sensor Arras and the Identification of Aliphatic Amines, Langmuir 24 (22), 2008, which is incorporated by reference herein in its entirety.

### Detection of Artificial Nose Response

Detector

In embodiments where a colorimetric sensor array 120 is utilized as the artificial nose technology, the color changes of the chemically responsive dyes 710 may be detected by any suitable optical or other detector 130. In embodiments pertaining to a colorimetric sensor array 120, a detector 130 may monitor the spectroscopic response, transmission response or reflectance response of the dyes 710 on the colorimetric sensing element at one or more wavelengths in a spatially resolved fashion so that all of the spots in the colorimetric sensor array 120 are individually imaged or addressed and the color of each spot is individually determined. For the purposes of the present disclosure, the terms color and colorimetric are intended to include wavelengths in the visible portion of the electromagnetic spectrum, as well as the invisible portion of the electromagnetic spectrum, e.g., infrared and ultraviolet. Color detection can be accomplished with an imaging spectrophotometer, a flatbed scanner, slide scanner, a video or CCD or CMOS digital camera, or a light source combined with a CCD or CMOS detector. Any still or video as well as analog or digital camera can be employed. Moreover, any imaging format can be used, e.g., RGB (red, green and blue) or YUV. Even the simple gray scale imaging can be used. In other embodiments utilizing other artificial nose technologies, a detector 130 or sensor may similarly be used provide a response of the detector indicative of the molecular interactions occurring at the detector 130 probe or other sensor.

The sensitivity of a colorimetric sensor array 120 is primarily a function of two factors, the ability of a dye 710 spot to change color when exposed to an analyte and the ability of the detector 130 to detect that color change. In some embodiments, an optical spectroscopic measurement system can divide the visible spectrum into as many as 500 individual bandpass windows whereas a three-color imaging system by definition contains only three such windows. An optical spectroscopic measurement system is therefore capable of detecting smaller color changes than can be detected by three-color imaging systems, effectively increasing the sensitivity of the entire cross-responsive sensing system. Accordingly, in particular embodiments of the present disclosure, an optical spectroscopic measurement system is employed as a detector 130. As used herein, optical spectroscopic measurement systems refer to any system that yields higher color resolution than a three-color imaging system. This can be an imaging spectrograph, fiber optic probe(s) coupled to a spectrograph, or other spectroscopic system.
Detection Process and System Setup

In some embodiments, the sample and container 200 are maintained at a constant temperature by an incubator. In some embodiments, the detector 130 may be incorporated into an incubator to allow the detector to continuously, or intermittently record the colorimetric response of the dyes 710 through window 270 while leaving the containers 200 undisturbed at constant temperature. After application of the VOCs contained in the urine may begin to react with the colorimetric sensor array 120.

Prior to application of a urine sample, a detector 130 may record an image of the presently loaded sensor array 120 as a control for later comparison and subtraction of color changes. Accordingly, this will allow the system to measure color changes based on variation from that particular array's initial color profile. In embodiments associated with other artificial nose technologies, the detector may record an initial reading for comparison to a later reading after introduction of a sample. After application of a urine sample to a sensor container 200, a detector 130 may at various intervals or after a set time interval, detect and record the colorimetric response of the dyes 710 or other detector 130 response. In some embodiments, software may be configured on server 150 or processing device 140 for automatically controlling the precise timing of detector 130 and recording of the data captured by detector 130. For example, the detector 810 may record an image ever minutes, 2, 3, 4, 5, 6, 7, 8, 9 or 10 minutes, or at intervals in-between, or at 20 or 30 minutes, or other suitable intervals. In some embodiments, the detector 130 may continuously record data from the colorimetric sensor array 120. The detector 130 may record images for an hour, 2, 3, 4, 5, 6, hours, or other suitable time frame. In some embodiments, the time frame may be selected based on when the color change rate is near homeostasis or has stopped reacting. In other embodiments, the color change may be stopped when a color change rate drops below a certain threshold.

### Data Processing

Data output from the detector 130 or other instruments associated with an artificial nose technology may then be stored and later processed for evaluation and diagnosis of the sample. A detector 130 may be incorporated into any suitable sensor or other instruments associated with an artificial nose technology system. The processing of the data may be performed on the processing device 140, server 150, or other computing device connected to the system. Various artificial nose technologies and systems may provide a response or an output indicative of the chemical or molecular interactions occurring at a sensor associated with the artificial nose technology. For example, many embodiments may utilize a detector 130 to detect changes after introduction of a urine sample containing VOCs.
Processing Detector Data for Colorimetric Sensor Array

In embodiments utilizing a colorimetric sensor array based artificial nose, a detector 130 may be utilized to detect optical changes in the array 120. In some embodiments, the detector 130 may only capture an image of the sensor array 120 before and at a single point in time after exposure of the dye 710 to the sample. In other embodiments, the detector 130 may capture images at various times or continuously after exposure of the sample to the dye 710. The color change differences before and at various points after introduction of the sample are used to classify or determine the properties of the sample. For example, when used in combination with colorimetric sensor array 120 and image analysis software, colorimetric differences can be generated by subtracting the values of dye images generated before and after exposure of the dye to a sample. In some embodiments, the colorimetric differences represent hue and intensity profiles for the array 120 in response to analytes contained in the sample. Thus for each image the detector 130 may extract a 240-dimensional 16-bit vector (R, G and B values if, for example, 80 indicators are used) before and after exposure. When used in accordance with the method of the present disclosure, a unique color change signature for the sample can be created which provides both qualitative recognition and quantitative analysis of volatile organic compounds present in the sample.

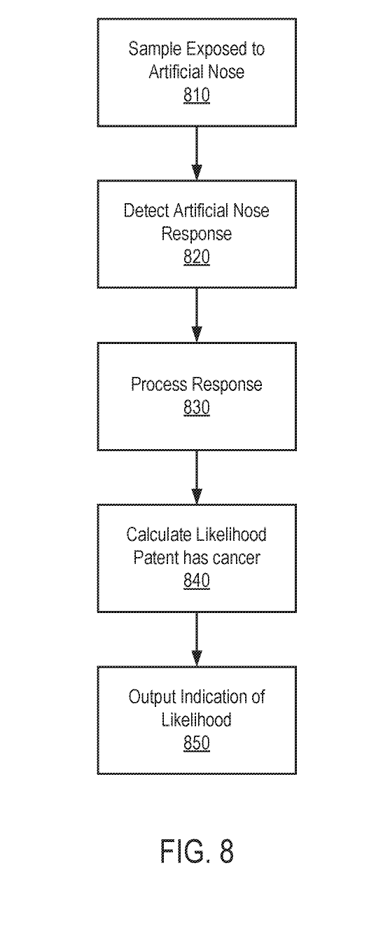

FIG. 8 illustrates an example of a process that certain or all of the steps of which may be implemented or controlled by a processing device 140, detector 130, associated database 160, server 150, and other electronic components that are communicating over a network. These computer or processor integrated components can automatically implement the illustrated process to provide an indication of whether a patient has lung cancer.

For example, first a sample may be exposed to an artificial nose 810. This may be implemented by a caregiver applying a sample inside a container 200 or a processing device 140 to open a door or other feature to allow exposure of the sample to the artificial nose 810 to begin. Exposure to the sample could be performed by any suitable method that allows the headspace gas to be exposed to, come in contact with, or come within gaseous proximity to the colorimetric array 120. This may include confining the colorimetric sensor array 120 and the urine sample or absorbent layer 290 within the same airtight container 200 or compartment. In some embodiments, bringing them within gaseous proximity may include storing the headspace gas from the urine sample and later exposing it to the colorimetric sensor array 120. Next, a response or image or several responses or images as described herein of the colorimetric array or other artificial nose 820 may be captured by a detector 130. In some embodiments, the detector 130 will first capture an image or several images of the colorimetric array or artificial nose 820 as a baseline prior to exposure of the colorimetric array 120 to the sample as described herein. Particularly, responses or readings from technologies may be taken before and after introduction of the sample to the artificial nose sensor or detector 130.

Next, the system may process the image data or other artificial nose response data 830 captured by the detector 130. As the processing 830 may be performed by any processing device 140 connected to the system. For example, the system may determine a colorimetric difference between the baseline image and images captured after exposure of the colorimetric sensor array 120 to the headspace gas from the urine sample.

Then, the system may calculate a likelihood a patient has cancer 840 or lung cancer based on the processing of the image or other detector 130 data 830. This may be performed by comparing the colorimetric difference determined in the processing 830 step to a database 160 to colorimetric differences associated with urine samples belong to patients with known ailments. For example, a statistical analysis may be performed using an HCA or PCA analysis (as described more fully herein) to determine the likelihood a sample indicates the associated patient has cancer based on comparisons to differences in the samples in the database that are known to have cancer or lung cancer.

The system may then output an indication of the likelihood 850 to a display associated with the system. The output of the indication may be percentage likelihood a patient has cancer, a threshold determination of whether the patient should have follow up screening or testing for cancer, or further testing to validate the results or other suitable indications. Outputting a percentage likelihood a patient has cancer may be advantageous, because prior diagnostic tests for lung cancer generally only provided a qualitative image or indication of whether a patient has cancer. The systems and methods disclosed herein may also be able to provide additional quantitative information regarding the diagnosis that may assist a patient in decision making.

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

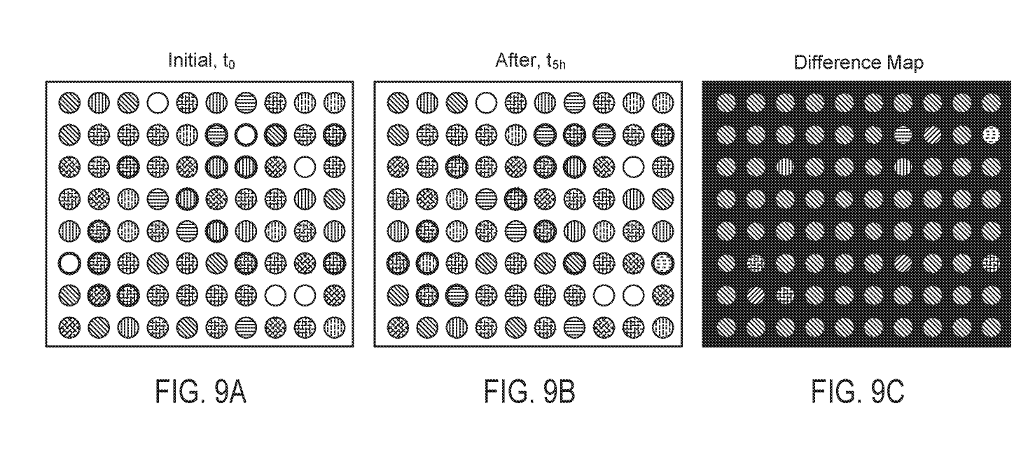

FIGS. 9A-9C are images from a colorimetric sensor array 120, showing the array before exposure to E. coli 25922 (FIG. 9A), after exposure to E. coli 25922 (FIG. 9B), and a difference map of these two images (FIG. 9C). The comparison data obtained from the difference map may include changes in red, green and blue values (ARGB) for each spot in the array 120. The changes in spectral properties that occur upon exposure to an analyte, and the resultant color difference map, can serve as a unique fingerprint for any analyte or mixture of analytes at a given concentration.

In the simplest case, an analyte can be represented by a single 3× vector representing the ARGB values for each colorant, where x is the number of colorants as set forth in equation (1). This assumes that equilibration is relatively rapid and that any irreversible reactions between analyte and colorant are slow relative to the initial equilibration time.

(1) Difference vector=ΔR1, ΔG1, ΔB1, ΔR2, ΔG2, ΔB2, . . . ΔRx, ΔGx, ΔBx

Alternatively, the temporal response of the analyte can be used to make rapid identification, preferably using a “time-stack vector” of ΔRGB values as a function of time. In equation (2), a time-stack vector is shown for an array of 36 colorants at times m, n, and finally z, all using the initial scan as the baseline for the differences in red, green and blue values:

(2) Time stack vector=ΔR1m, ΔG1m, ΔB1m, ΔR2m, ΔG2m, ΔB2m, −ΔR36m, ΔG36m, ΔB36m, . . . ΔR1n, ΔG1n, ΔB1n, . . . ΔR36m, ΔG36m, ΔB36m, . . . ΔR36z, ΔG36z, ΔB36z

Accordingly, each analyte response can be represented digitally as a vector of dimension 3xz, where x is the number of colorants and z is the number of scans at different times.
Statistical Analysis

Quantitative comparison of such difference vectors can be made simply by measuring the Euclidean distance in the 3×z space. Such vectors may then be treated by using chemometric or statistical analyses, including principal component analysis (PCA), hierarchical cluster analysis (HCA) and linear discriminant analysis. Statistical methods suitable for high dimensionality data are preferred. As an example, HCA systematically examines the distance between the vectors that represent each colorant, forming clusters on the basis of the multivariate distances between the analyte responses in the multidimensional ARGB color space using the minimum variance (“Ward's”) method for classification. A dendrogram can then be generated that shows the clustering of the data from the Euclidean distances between and among the analyte vectors, much like an ancestral tree.

### Lung Cancer Metabolism

Biomarkers for lung cancer from blood, saliva, and urine have been identified, including the following: proteins, tumor antigens, anti-tumor antibodies, cell type-specific peptides, metabolic products and epigenetic phenomena such as hyper-methylated DNA, NRA, and the expression of specific genes. However, to date, none of these biomarkers has had the adequate sensitivity, specificity, and reproducibility to be utilized in an effective diagnostic test.

However, potential effective biomarkers for lung cancer may include low molecular weight volatile organic compounds, which can be detected on the urine of patients. This evidence for lung cancer metabolism is expected to manifest as a characteristic concentration profile covering dozens of metabolic VOCs. For example, the volatile organic compounds dimethyl succinate, 2-pentanone, phenol, 2-methylpyrazine, 2-hexanone, 2-butanone and acetophenone, among others, have been found in increased concentrations in the urine headspace of mice implanted with lung cancer cell lines. (Hani, et al., Analysis of volatile organic compounds released from human lung cancer cells and from the urine of tumor-bearing mice, Cancer Cell International, 2012, 12:7).

### Example

In order to determine whether any of these or other volatile organic compounds can be detected using a colorimetric sensor array 120 in order to detect or diagnose lung cancer, the following experiment was conducted.
Methods

149 urine samples were collected included 43 controls and 57 cancer urine samples with adenocarcinoma (34) and squamous cell carcinoma (18). Additionally, five cancer subjects with histology other than adenocarcinoma and squamous cell were also included in order to establish the capability to overall distinguish cancer vs. control. Additional clinical information, such as age, gender, smoking history and COPD, was provided, and included in the analysis. Summary of subject demographics is given in Table 4. There were 49 additional urine samples collected from patients with indeterminate nodules, but they were excluded from this preliminary analysis.
Urine Assay Methods

Each urine sample was divided into multiple aliquots of 1.5 mL and stored at −80 degrees Celsius. For each test, a frozen aliquot was slowly warmed to room temperature until completely thawed, then 200 μL of urine was loaded onto a blotting paper or absorbent layer 290 embedded in the container 200, as shown in Table 4. A processing device 140 and associated software automatically control a flatbed scanner (Epson V600) and image the sensor array 120 at 3-minute intervals for duration of 4 hours. Both the scanner and sensor 120 were kept inside an incubator at 37 degrees Celsius for the duration of the test to minimize the variation in room temperature.

TABLE 4
Baseline demographics.

| Histology | n | Gender | Age (mean) | Smoking | COPD |
|---|---|---|---|---|---|
| Adenocarcinoma | 34 | 17 M, 17 F | 45-86 (68) | 5 Never, 26 Former, | 1 |
| | | | | 3 Current | |
| Squamous Cell | 18 | 14 M, 4 F | 43-83 (63) | 12 Former, 6 Current | 8 |
| Large Cell | 1 | 1 F | 59 | 1 Former | 1 |
| Small Cell | 3 | 1 M, 2 F | 60-77 (67) | 1 Former, 2 Current | 1 |
| Adeno/Squamous | 1 | 1 M | 65 | 1 Current | 0 |
| Cell | | | | | |
| Control | 43 | 20 M, 23 F | 41-78 (63) | 1 Never, 27 Former, 15 | 16 |
| | | | | Current | |
| Total | 100 | 53 M, 47 F | | 6 never, 67 former, 27 current | 27 |

Additives

Chemical additives can differentially liberate urine VOCs. In addition to measuring sensor array 120 responses to neat urine headspace, the inventors added various additives to optimize the discrimination between lung cancer patients and healthy controls. For instance, alkaline solution (1.0 M NaOH) was added to convert amines into their volatile free base form, and acid solution (1.0 M p-toluenesulfonic acid) to protonate and release organic acids into the headspace. Sodium chloride was added to the saturation point to maximize the “salting-out” effect to promote the release of hydrophobic VOC's. Also, we have used a pre-oxidation tube to oxidize the urine VOCs to enhance sensor array 120 sensitivity to hydrocarbons and aromatics. In some embodiments, a sensor array 120 sensitivity towards hydrocarbons substantially improves when these compounds are pre-oxidized with sulfochromic acid. Non-volatile additives were selected to avoid any potential interference with the urine volatiles. Additives were mixed with urine in a 1:1 volume ratio, and every biological sample was tested in duplicate with each of the six additive conditions: 1) neat urine, 2) urine+1 M tosic acid, 3) urine +1M NaOH, 4) NaCl, 5) desiccant and chromic acid, and 6) sodium citrate buffer.
Data Processing

Data from the scanned images was processed by extracting the red, green and blue (RGB) values from each one of the 73 spot indicators incorporating chemically responsive dyes 710 (73×3=219 elements per image). By processing all the images collected throughout the test duration (4 hours at 3 minutes imaging interval, 20×4=80 images), a matrix of 219×80 was constructed representing the time response of each indicator. Since each urine sample was run with six additive conditions, each vector file had possible 219×80×6=105,120 dimensions. Only four time points were extracted out of the 80 (equivalent to sampling the indicators colors change every 1 hour). Hence, 5,256 features were used for data analysis.

To normalize the data, a baseline correction was performed, by subtracting the initial color (R, G and B) of each indicator from subsequent time series values. The statistical analysis was all performed in R using standard available packages. The dimensionality of the feature set was further reduced by removing any constant features, and ran a pairwise t-test to eliminate features with no statistical significance (p>0.05), and then an elastic net (alpha=0.2) was applied to further reduce the surviving features. By repeating this process <150 iterations most significant features in the dataset were extracted.

To construct a classifier, the dataset was partitioned into non overlapping training set and test set using bootstrap stratified sampling. A support vector machine classifier (SVM) was trained on the reduced features in the training set, and its classification performance on the test set was evaluated. Validated classifier performance was averaged over 100 bootstrap partitions. Since the total number of samples (N=100) is relatively small compared to the sensor dimensionality, the results were verified by randomly permuting the class labels for all the trials. The randomly permuted labels performed significantly worse than the true labels as summarized in Table 5.

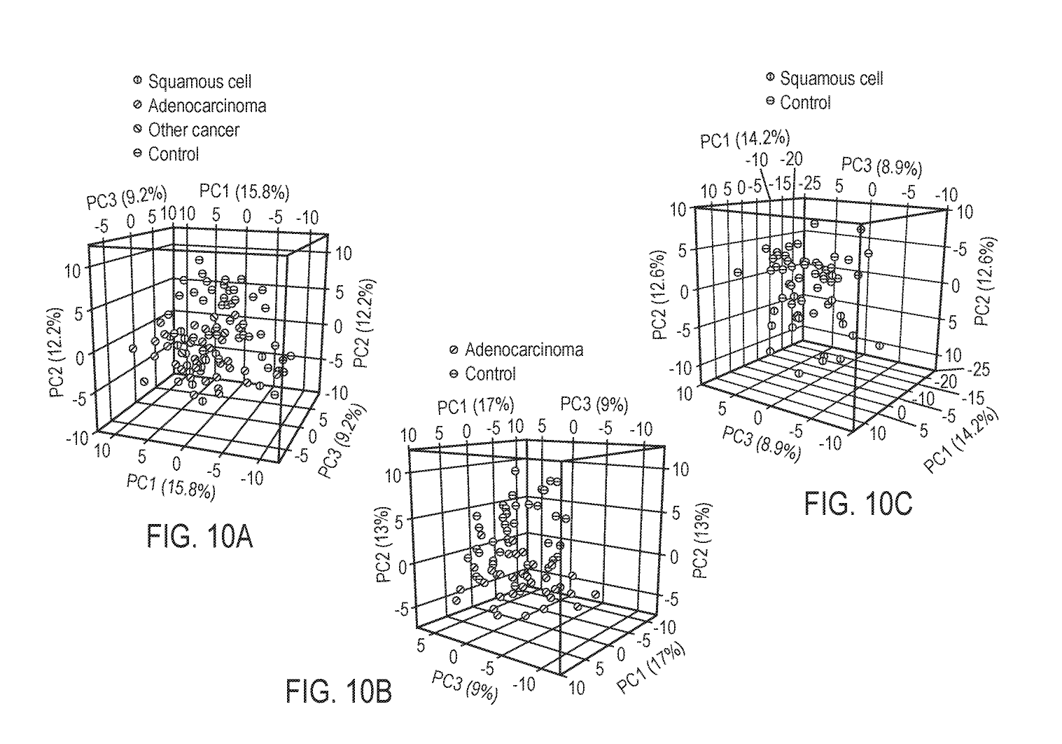

Although this preliminary dataset contains only 100 biological samples, the sensor array 120 technology showed very promising results, with plenty of room to improve accuracy by optimizing the indicators and additives. Multiple channels already exhibit distinct kinetic profiles in response to adenocarcinoma or squamous cell samples. Class separation is visible in the PCA score plot as shown in FIGS. 10A-10C. FIGS. 10A-10C illustrate PCA score plots performed on selected feature space. FIG. 10A illustrates cancer vs. control, FIG. 10B illustrates adenocarcinoma vs. control and FIG. 10C illustrates squamous cell vs. control.

TABLE 5
Accuracy, Sensitivity and specificity of lung cancer.

| | Sensitivity | Specificity | Accuracy |
|---|---|---|---|
| Cancer (57) Vs. Control (43) | 82.5% | 82% | 83.5% |
| Adenocarcinoma (34) Vs. Control | 84.7% | 81.9% | 87% |
| (43) | | | |
| Squamous Cell Carcinoma (18) Vs. | 93.6% | 96% | 87.8% |
| Control (43) | | | |

### Further Applications

Although the present application has been primarily described with reference to diagnosis of lung cancer based on experimental validation, these devices and methods can be applied to diagnose additional diseases in addition to lung cancer. For instance, the container 200 and sensor array 120 may be applied to identify other types of cancer other than lung cancer, which will likely have unique VOC profiles that may be identified using the sensor array 120 or other artificial nose technology.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

1. A method for determining an indication of whether a patient has cancer comprising:
exposing an artificial nose to the headspace gas from a patient's urine sample; and
determining an indication of whether the patient has cancer based on the response of the artificial nose to volatile organic compounds contained in the headspace gas from the patient's urine.

2. The method of claim 1, wherein the artificial nose is a colorimetric sensor array.

3. The method of claim 1, wherein exposing comprises placing the urine sample together inside a container with the artificial nose.

4. The method of claim 1, wherein exposing comprises placing the artificial nose in gaseous communication with the urine sample.

5. The method of claim 1, wherein chemical additives are mixed with the urine to differentially liberate urine VOCs.

6. The method of claim 5, wherein the chemical additives are one or more of the following:
tosic acid, NaOH, NaCl, desiccant and chromic acid and sodium citrate buffer.

7. The method of claim 1, wherein a chemical oxidizer is used to react with urine VOCs to facilitate detection by an artificial nose.

8. The method of claim 7, wherein the chemical oxidizer is a chromic acid.

9. The method of claim 1, wherein the exposing comprises collecting the headspace gas from the urine sample and transferring it to an enclosed container containing the artificial nose.

10. The method of claim 1, wherein the cancer is lung cancer.

11. The method of claim 1, wherein the cancer is selected from the group consisting of prostate cancer, colorectal cancer, kidney (renal cell) cancer, bladder cancer, breast cancer, non melanoma skin cancers, endometrial cancer, leukemia, melanoma, non-hodgkin lymphoma, pancreatic cancer and thyroid cancer.

12. The method of claim 1, wherein the determining the indication of whether the patient has cancer includes identifying the subtype of cancer.

13. The method of claim 1, wherein the determining the indication of whether the patient has cancer based on the response of the artificial nose further comprises capturing a baseline response of the artificial nose before exposure to the headspace gas from the urine sample and at least one response after exposure to the headspace gas from the urine sample.

14. The method of claim 11, further comprising processing the baseline response and at least one response after exposure to determine a difference between the baseline and after exposure responses.

15. The method of claim 11, further comprising comparing the artificial nose response using statistical analysis to a database of responses associated with patients that are known to have lung cancer.

16. The method of claim 1, wherein the determining an indication of whether a patient has cancer further comprises determine a probability a patient has cancer.

17. The method of claim 1, wherein the at least one response after exposure comprises a plurality of responses taken at regular intervals.

18. The method of claim 11, wherein the at least one response after exposure comprises recording a continuous response.

19. A method for diagnosing cancer of a patient comprising:
placing a urine sample from a patient inside a container that also contains an artificial nose; and
determining an indication of whether the patient has cancer based on the response of the artificial nose to volatile organic compounds emitted by the urine sample.

20. The method of claim 19 wherein the artificial nose is a colorimetric sensor array.

21. The method of claim 19 wherein the cancer is lung cancer.

22. The method of claim 19 wherein, wherein the container is a compartment inside a multi-compartment tray.

23. The method of claim 19 wherein the determining an indication of whether the patient has cancer includes identifying the subtype of cancer of the patient.

24. The method of claim 19, wherein chemical additives are mixed with the urine to differentially liberate urine VOCs.

25. The method of claim 24, wherein the chemical additives are one or more of the following: tosic acid, NaOH, NaCl, desiccant and chromic acid and 6 sodium citrate buffer.

26. The method of claim 19, wherein a chemical oxidizer is used to react with urine VOCs to facilitate detection by an artificial nose.

27. The method of claim 26, wherein the chemical oxidizer is a chromic acid.

28. The method of claim 19, wherein the determining an indication of whether the patient has cancer based on the response of the colorimetric sensor further comprises capturing a baseline image of the colorimetric sensor array before placing the urine sample in the container and at least one dynamic image after placing the urine sample in the container.

29. The method of claim 28, further comprising processing the baseline image and at least one dynamic image to determine a colorimetric difference between the baseline and after exposure images.

30. The method of claim 29, further comprising comparing the colorimetric difference to a database of colorimetric differences associated with patients that are known to have lung cancer.

31. The method of claim 30, wherein the comparing step is performed using statistical analysis.

32. The method of claim 19, wherein the determining an indication of whether a patient has cancer further comprises determine a probability a patient has cancer.

33. The method of claim 28, wherein the at least one dynamic response comprises a plurality of images taken at regular intervals.

34. The method of claim 28, wherein the at least one dynamic response comprises recording a continuous image for a set interval of time.

35. A device for diagnosing the lung cancer status of a patient based on the patient's urine comprising:
a container having a cavity and a window that allows transmission of radiation into and out of the cavity;
a colorimetric sensor array disposed inside the cavity of the container;
a reversibly sealable opening that provides access to the cavity for depositing of a urine sample within the cavity; and
an absorbent material positioned between the colorimetric sensor array and the opening for application of the urine sample.

36. The device of claim 35, wherein the absorbent material divides the cavity into an array sub-cavity and a sample sub-cavity, and the absorbent material is sealed around the edges to prevent airflow between the array sub-cavity and the sample sub-cavity except for airflow travelling through the absorbent material.

37. The device of claim 36, wherein the absorbent material is sealed around the edges using a ring gasket.

38. The device of claim 35, wherein the cavity is free of culture media.

39. The device of claim 35, wherein the opening is created by reversibly unfastening a sample portion of the container.

40. The device of claim 35, wherein the opening is reversibly sealed using a stopper.

41. The device of claim 35, wherein the colorimetric sensor array is shielded from urine with a barrier.

42. The device of claim 41, wherein the barrier is a cover sheet.

43. The device of claim 35, wherein a spacer creates a gap between the colorimetric sensor array and the window.

44. A device comprising:
a reversibly sealable container defining a cavity and having a window for transmission of UV, visible and infrared light into and out of the container;
an absorbent material positioned in the cavity;
a colorimetric sensor array configured to be positioned proximate the absorbent material and the window when the container is sealed; and
wherein the container is free of culture media, and the absorbent is arranged to accept a biological sample such that only volatile organic compounds from the biological sample contact the colorimetric sensor array when the container is sealed.

45. A method for analyzing a urine sample from a patient to determine whether the patient has lung cancer comprising:
capturing an baseline image of a colorimetric sensor array;
capturing at least one additional image of the colorimetric sensor array after the sensor array has been exposed to headspace gas from a urine sample from a patient; and
processing the baseline and at least one additional image to calculate a likelihood a patient has cancer.

46. The method of claim 45, further comprising outputting an indication of whether the patient has cancer.

47. The method of claim 46, wherein the indication is a percentage chance the patient has cancer.

48. The method of claim 46, wherein the indication is a threshold determination of whether the patient is likely to have cancer.

49. The method of claim 45, wherein the processing step further comprises determining a colorimetric difference between the baseline image and the at least one additional image.

50. The method of claim 49, wherein the difference is compared to colorimetric data from urine samples of patients known to have cancer stored in a database.
